(19) United States
(12) Patent Application Publication
(10) Pub. No.: US 2019/0144958 A1
(43) Pub. Date: May 16, 2019

(54) CULTURED LEATHER AND PRODUCTS MADE THEREFROM

(71) Applicant: Provenance Biofabrics, Inc., La Jolla, CA (US)

(72) Inventors: Christian EWTON, Los Angeles, CA (US); Michalyn ANDREWS, Pacific Palisades, CA (US)

(21) Appl. No.: 16/099,130

(22) PCT Filed: May 5, 2017
(86) PCT No.: PCT/US2017/031388

§ 371 Date: Nov. 5, 2018

Related U.S. Application Data
(60) Provisional application No. 62/332,881, filed on May 6, 2016.

(51) Int. Cl. C14C 13/02; C12N 5/071; C08J 3/24; C12N 5/077; C12N 5/074; C12Q 1/686; C12Q 1/6806; B32B 9/02; C12Q 1/6876

(52) U.S. Cl. CPC C14C 13/02; C12N 5/0629; C08J 3/24; C12N 5/0656; C12Q 2563/185; C12Q 1/686; C12Q 1/6806; B32B 9/025; C12Q 1/6876; C12N 5/0696

(57) ABSTRACT

A cultured leather comprising a plurality of layers each layer comprising cells and extracellular collagen, wherein each layer of the plurality of layers is coupled to an adjacent layer by a nucleotide adhesion molecule, wherein the cultured leather comprises a moisture content of less than 40%.

### CROSS-REFERENCE TO RELATED APPLICATIONS

This application claims the benefit of U.S. Provisional Application No. 62/332,881 filed on May 6, 2016, the contents of which is hereby incorporated herein by reference.

### BACKGROUND OF THE INVENTION

Leather, made from animal hide, has existed for millennia. Originally these hides were utilized from animals hunted for sustenance, and leather was one readily accessible source of highly durable and functional fabric. Today, however, leather is a luxury good utilized mainly for fashion and luxury appeal. The current procurement of leather is objectionable on both sustainability and ethical grounds. At 65-150 kg of CO₂/m² the carbon footprint of bovine leather is much more significant than other fabrics. Additionally, the production of leather requires growing and killing animals which many find ethically objectionable. Disclosed herein are cultured leather products and methods of making cultured leather products that address these current shortcomings with leather.

### SUMMARY OF THE INVENTION

Disclosed herein, in certain embodiments, are cultured “bioleathers” and products made therefrom. In general, the cultured leather of this disclosure are made by culturing skin cells derived from a suitable animal, such as a cow, and subjecting the cells to one or more processing steps that results in a useable leather product. The methods and products of this disclosure solve many problems currently associated with the production of cultured leather, primarily generating sufficiently large sheets of cells held together with sufficient strength to allow for processing into leather. This is accomplished without the destruction of animals or the large carbon footprint of traditional leather.

In certain embodiments, disclosed herein, is a cultured leather comprising, a plurality of layers each layer comprising cells and extracellular collagen, wherein each layer of the plurality of layers is coupled to an adjacent layer by a nucleotide adhesion molecule, wherein the cultured leather comprises a moisture content of less than 40%. In certain embodiments, the cells comprise fibroblasts. In certain embodiments, the cells comprise keratinocytes. In certain embodiments, the cells comprise induced pluripotent stem cells (iPS cells or iPSCs). In certain embodiments, some or all of the cells originate from any one or more of a mammal, a bird, a fish, a reptile, or an amphibian. In certain embodiments, the extracellular collagen is cross-linked. In certain embodiments, the nucleotide adhesion molecule comprises: a membrane anchoring region comprising a long alkyl chain of at least 12 carbon atoms; and a polynucleotide having a membrane distal end and a membrane proximal end, wherein said polynucleotide is conjugated to said membrane anchoring region at the membrane proximal end wherein said polynucleotide is at least 50 nucleotides and comprises: a linker region comprising a contiguous stretch of at least about 20 nucleotides; and a membrane distal adhesion region comprising at least 10 nucleotides and positioned distal to the linker region, wherein the linker region is not hybridizable to the membrane distal adhesion region. In certain embodiments, the nucleotide adhesion molecule comprises a DNA signature, wherein the DNA signature is able to be amplified after the tanning process. In certain embodiments, the moisture content is between 10% and 30%. In certain embodiments, the tensile strength equals or exceeds 25 kgf/cm². In certain embodiments, the cultured leather is less than 100 millimeters thick. In certain embodiments, described herein are consumer products comprising the cultured leather. In certain embodiments, the consumer product is a wallet. In certain embodiments, the consumer product is a purse. In certain embodiments, the consumer product is a watch band. In certain embodiments, the consumer product is a jacket. In certain embodiments, the consumer product is a shoe. In certain embodiments, the consumer product is a mobile phone case. In certain embodiments, the consumer product is a brief case. In certain embodiments, the consumer product is a piece of luggage. In certain embodiments, the consumer product is a glove. In certain embodiments, the consumer product is a belt. In certain embodiments, the consumer product is a pair of pants.

In certain embodiments, described herein, is a method of preparing a cultured leather comprising: culturing a plurality of layers, each layer comprising cells, wherein each layer of the plurality of layers is coupled to an adjacent layer by a nucleotide adhesion molecule; and contacting the plurality of layers with at least one tanning agent; wherein the resulting cultured leather comprises a moisture content of less than 40%. In certain embodiments, the cells comprise fibroblasts. In certain embodiments, the cells comprise keratinocytes. In certain embodiments, the cells comprise induced pluripotent stem cells (iPSC). In certain embodiments, some or all of the cells originate from any one or more of a mammal, a bird, a fish, a reptile, or an amphibian. In certain embodiments, the collagen is cross-linked by the tanning agent. In certain embodiments, the tanning agent comprises chromium. In certain embodiments, the tanning agent comprises a vegetable tannin. In certain embodiments, the nucleotide adhesion molecule comprises: a membrane anchoring region comprising a long alkyl chain of at least 12 carbon atoms; and a polynucleotide having a membrane distal end and a membrane proximal end, wherein said polynucleotide is conjugated to said membrane anchoring region at the membrane proximal end wherein said polynucleotide is at least 50 nucleotides and comprises: a linker region comprising a contiguous stretch of at least about 20 nucleotides; and a membrane distal adhesion region comprising at least 10 nucleotides and positioned distal to the linker region, wherein the linker region is not hybridizable to the membrane distal adhesion region. In certain embodiments, the nucleotide adhesion molecule comprises a DNA signature, wherein the DNA signature is able to be amplified after the tanning process. In certain embodiments, the moisture content of the resulting cultured leather is between 10% and 30%. In certain embodiments, the tensile strength of the resulting cultured leather equals or exceeds 25 kgf/cm². In certain embodiments, the method further comprises using the cultured leather in the manufacture of a consumer product. In certain embodiments, the consumer product is a wallet. In certain embodiments, the consumer product is a purse. In certain embodiments, the consumer product is a watch band. In certain embodiments, the consumer product is a jacket. In certain embodiments, the consumer product is a shoe. In certain embodiments, the consumer product is a mobile phone case. In certain embodiments, the consumer product is a brief case. In certain embodiments, the consumer product is a piece of luggage. In certain embodiments, the consumer product is a glove. In certain embodiments, the consumer product is a belt. In certain embodiments, the consumer product is a pair of pants.

### BRIEF DESCRIPTION OF THE DRAWINGS

FIG. 1 shows a simplified scheme for a method of culturing cells to be used in the manufacture of cultured leather. In this scheme, two cell layers are held together by two different nucleotide adhesion molecules with distinct adhesion sequences (grey and black triangles) that are held together by complementary base pairing. These sequences are joined to the membrane anchoring region (grey box) by a linker region (black line). In certain embodiments, more than 2 layers are fabricated in this manner. In certain embodiments, the layers are cellularly or compositionally distinct.

FIG. 2 illustrates a design for a pair of pants optionally fabricated from the cultured leather.

FIG. 3 illustrates a design for a handbag optionally fabricated from the cultured leather.

FIG. 4 illustrates a design for a briefcase optionally fabricated from the cultured leather.

FIG. 5 illustrates a design for a suitcase optionally fabricated from the cultured leather.

FIG. 6 illustrates a design for a women's pump optionally fabricated from the cultured leather.

FIG. 7 illustrates a design for a men's loafer optionally fabricated from the cultured leather.

FIG. 8 illustrates a design for a belt optionally fabricated from the cultured leather.

FIG. 9 illustrates a design for a wallet optionally fabricated from the cultured leather.

FIG. 10 illustrates a design for a pair of gloves optionally fabricated from the cultured leather.

FIG. 11 illustrates a design for a watch band optionally fabricated from the cultured leather.

FIG. 12 illustrates a design for a sunglasses case optionally fabricated from the cultured leather.

FIG. 13 illustrates a design for a steering wheel cover optionally fabricated from the cultured leather.

FIG. 14 illustrates a design for an airplane seat optionally fabricated from the cultured leather.

FIG. 15 illustrates a design for a football optionally fabricated from the cultured leather.

FIG. 16 illustrates a design for a dining chair optionally fabricated from the cultured leather.

FIG. 17 illustrates a design for a journal optionally fabricated from the cultured leather.

### DETAILED DESCRIPTION OF THE INVENTION

### Certain Definitions

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

As used herein “cultured leather,” also called “bioleather” herein, refers to leather that is fabricated from individual living cells originated from an animal, which is not killed to obtain the cells. In certain embodiments, cultured leather has been grown using standard tissue/cell culture methods and media.

As used herein “natural leather” refers to leather that is traditionally fabricated from animal skin or hides.

As used herein “substrate” refers to a support for cells, cell layers, tissues, or cultured leather, which is non-integral to a cell layer, a tissue, or the cultured leather and is temporary, e.g., removed during or subsequent to fabrication. A substrate, in some embodiments, is permeable to tissue/cell culture medium. A substrate differs from pre-made scaffold or other support material, which is integrated into a cell layer, a tissue, or leather product.

### Cultured Leather

Disclosed herein, in certain embodiments, is a cultured leather comprising, a plurality of layers each layer comprising cells and extracellular collagen, wherein each layer of the plurality of layers is coupled to an adjacent layer by a nucleotide adhesion molecule, wherein the cultured leather comprises a moisture content less than 40%. Disclosed herein, in certain other embodiments, is a cultured leather comprising, a plurality of layers each layer comprising cells, wherein each layer of the plurality of layers is coupled to an adjacent layer by a nucleotide adhesion molecule, wherein the cultured leather comprises a moisture content less than 40%. Disclosed herein, in certain other embodiments, is a cultured leather comprising, a layer of cells and extracellular collagen, wherein the cultured leather comprises a moisture content less than 40%.

In certain embodiments, the cultured leather refers to any leather that is made in a laboratory or production facility from cultured cells, and not sourced from an animal that is dead. In certain embodiments, the cells originate from an animal, and are then immortalized or expanded using standard tissue culture techniques. In certain embodiments, the cells originate from a skin sample obtained from an animal that is not killed. In certain, embodiments, cultured leather does not comprise purely synthetic leather-like or faux leather products. Synthetic forms of leather include those made from vinyl, polyvinylchloride, polyurethane, or the like.

In various embodiments, the cultured leather differs from naturally occurring tissues and leather in many ways. In certain embodiments, the cultured leather lacks hair follicles. In certain embodiments, the cultured leather lacks hair or fur. In certain embodiments, the cultured leather lacks sebocytes. In certain embodiments, the cultured leather lacks vasculature. In certain embodiments, the cultured leather further comprises a non-naturally occurring amplifiable DNA molecule. In certain embodiments, the cultured leather comprises cells taken from at least two different donors. In certain embodiments, the cultured leather is chimeric and comprises cells from two different species. In certain embodiments, the cultured leather comprises fibroblasts of non-dermal origin.

### Layers and Architecture

In certain embodiments, described herein the cultured leather comprises a plurality of layers. In certain embodiments, the plurality of layers comprises at least 1, 2, 3, 4, 5, 6, 7, 8, 9, 10 or more layers. In certain embodiments, the plurality of layers comprises at least 10, 20, 30, 40, 50, 60, 70, 80, 90, 100 or more layers, including increments therein. In certain embodiments, any layer of the plurality of layers comprises a single cell type. In certain embodiments, any layer of the plurality of layers comprises two different cell types. In certain embodiments, any layer of the plurality of layers comprises three different cell types. In certain embodiments, any layer of the plurality of layers comprises four or more different cell types. In certain embodiments, any layer of the plurality of layers consists essentially of a single cell type. In certain embodiments, any layer of the plurality of layers consists essentially of two different cell types. In certain embodiments, any layer of the plurality of layers consists essentially of three different cell types. In certain embodiments, any layer of the plurality of layers consists essentially of four or more different cell types. In certain embodiments, a layer is a single cell thick. In certain embodiments, a layer is greater than a single cell thick. In certain embodiments, a layer is 1, 2, 3, 4, 5, 6, 7, 8, 9, 10 or more cells thick. In certain embodiments, a layer is at least 10, 20, 30, 40, 50, 60, 70, 80, 90, 100 or more cells thick, including increments therein.

In certain embodiments, the cultured leather is chimeric comprising cells of a different cell type, different species, or different breeds of the same species. In certain embodiments, any of the plurality of layers of cultured leather is chimeric comprising a mixture of cells of a different cell type, different species, or different breeds of the same species. In certain embodiments, at least one layer is distinct from an adjacent layer. In certain embodiments, the cultured leather lacks pre-made scaffold or other support material, which is integrated into the cultured leather. In certain embodiments, at least one layer comprises cells from a different animal species of an adjacent layer. In certain embodiments, at least one layer comprises cells from a different breed of the same species of an adjacent layer.

In certain embodiments, the cultured leather is at least 0.1 millimeters thick. In certain embodiments, the cultured leather is at least 0.2 millimeters thick. In certain embodiments, the cultured leather is at least 0.5 millimeters thick. In certain embodiments, the cultured leather is at least 1 millimeter thick. In certain embodiments, the cultured leather is at least 2 millimeters thick. In certain embodiments, the cultured leather is at least 5 millimeters thick. In certain embodiments, the cultured leather is at least 10 millimeters thick. In certain embodiments, the cultured leather is less than 100 millimeters thick. In certain embodiments, the cultured leather is less than 10 millimeters thick. In certain embodiments, the cultured leather is less than 5 millimeters thick. In certain embodiments, the cultured leather is less than 2 millimeters thick. In certain embodiments, the cultured leather comprises at least 1 square centimeter of surface area. In certain embodiments, the cultured leather comprises at least 10 square centimeters of surface area. In certain embodiments, the cultured leather comprises at least 100 square centimeters of surface area. In certain embodiments, the cultured leather comprises at least 1000 square centimeters of surface area.

### Cell Types

In certain embodiments, the cultured leather described herein, is made from any suitable cell type. In certain embodiments, the skin cell is a cell of the dermis. In certain embodiments, the skin cell is a cell of the epidermis. In some embodiments, the skin cell is a cell of the basal membrane. In certain embodiments, the cultured leather described herein, is made from a skin cell. In certain embodiments, the skin cell is a keratinocyte. In certain embodiments, the skin cell is a fibroblast. In certain embodiments, the skin cell is a pigment cell. In certain embodiments, the skin cell is a melanocyte. In certain embodiments, the skin cell is a keratinocyte. In certain embodiments, the cultured leather described herein comprises pluripotent stem cells. In certain embodiments, the cultured leather described herein comprises pluripotent stem cells that have been differentiated into a skin-like cell. In certain embodiments, the differentiation of pluripotent stem cells yields a heterogeneous cell population. In certain embodiments, the differentiation of pluripotent stem cells yields vascular cells. In certain embodiments, the differentiation of the pluripotent stem cell yields cells of sebaceous glands. In certain embodiments, the differentiation of the pluripotent stem cell yields cells of hair follicles. In certain embodiments, the differentiation of the pluripotent stem cell yields cells of the nervous system. In certain embodiments, the differentiation of the pluripotent stem cell yields adipocytes. In certain embodiments, the differentiation of the pluripotent stem cell yields fibroblasts. In certain embodiments, the differentiation of the pluripotent stem cell yields a combination of one or more the cell types described herein.

In certain embodiments, the cultured leather comprises cells derived from at least two different donor animals. In certain embodiments, cultured leather does not comprise adipose cells or adipose tissue. In certain embodiments, cultured leather does not comprise muscle cells or tissue. In certain embodiments, cultured leather does not comprise sebocytes.

In certain embodiments, the plurality of layers comprises one or more layers of keratinocytes adjacent to one or more layers of fibroblasts. In certain embodiments, any of the plurality of layers comprises a mixture of fibroblasts and keratinocytes. In certain embodiments, the mixture comprises at least about 95%, 90%, 85%, 80%, 75, 70%, 65%, 60%, 55%, 50%, 45%, 40%, 35, 30%, 25%, 20%, 15%, 10%, or 5% keratinocytes, including increments therein. In certain embodiments, the mixture comprises at least about 95%, 90%, 85%, 80%, 75, 70%, 65%, 60%, 55%, 50%, 45%, 40%, 35, 30%, 25%, 20%, 15%, 10%, or 5% fibroblasts, including increments therein.

In a particular embodiment, the plurality of layers comprises one or more epidermis-like layers. In further embodiments, an epidermis-like layer comprises about 80%, about 85%, about 90, about 95%, or about 98% keratinocytes and squamous epithelial cells, including increments therein. In a particular embodiment, the plurality of layers comprises one or more dermis-like layers. In further embodiments, a dermis-like layer comprises a mixture of fibroblast, macrophages, and adipocytes. In a particular embodiment, one or more layers comprise extracellular matrix components, such as collagen, elastin, fibronectin, and/or laminin, either secreted by the cells or introduced in the fabrication process.

The cells used in the cultured leather described herein originate from a biopsy of animal tissue without the animal being harmed or killed. In certain embodiments, the cultured leather comprises cells that originate from a mammal (e.g., cow), a bird (e.g., ostrich) a fish (e.g., shark), a reptile (e.g., snake), an amphibian, or a combination thereof.

### Collagen

In certain embodiments, the cultured leather comprises collagen. In certain embodiments, the cultured leather comprises collagen that has been secreted by cells maintained in culture. In certain embodiments, the cultured leather comprises collagen that is crosslinked. In certain embodiments, the cultured leather comprises extracellular collagen. In certain embodiments, the cultured leather comprises extracellular collagen that is crosslinked. In certain embodiments, the cultured leather comprises about 25% to about 75% collagen by weight. In certain embodiments, the cultured leather comprises at least about 25% collagen by weight. In certain embodiments, the cultured leather comprises at least about 30% collagen by weight. In certain embodiments, the cultured leather comprises at least about 35% collagen by weight. In certain embodiments, the cultured leather comprises at least about 40% collagen by weight. In certain embodiments, the cultured leather comprises at least about 45% collagen by weight. In certain embodiments, the cultured leather comprises at least about 50% collagen by weight. In certain embodiments, the cultured leather comprises at least about 55% collagen by weight. In certain embodiments, the cultured leather comprises at least about 60% collagen by weight. In certain embodiments, the cultured leather comprises at least about 65% collagen by weight. In certain embodiments, the cultured leather comprises at least about 70% collagen by weight. In certain embodiments, the cultured leather comprises no more than about 65% collagen by weight. In certain embodiments, the cultured leather comprises no more than about 60% collagen by weight. In certain embodiments, the cultured leather comprises no more than about 50% collagen by weight. In certain embodiments, the cultured leather comprises no more than about 45% collagen by weight. In certain embodiments, the cultured leather comprises no more than about 40% collagen by weight. In certain embodiments, the cultured leather comprises no more than about 35% collagen by weight. In certain embodiments, the cultured leather comprises no more than about 30% collagen by weight. In certain embodiments, the collagen has been crosslinked by a tanning agent. In certain embodiments, the tanning agent is a vegetable tannin. In certain embodiments, the vegetable tannin is from chestnut, oak, redoul, tanoak, hemlock, quebracho, mangrove, wattle (acacia; see catechol), and myrobalans from Terminalia spp., such as Terminalia chebula. In certain embodiments, the tannin is a chemical tannin. In certain embodiments, the chemical tannin is a chromium compound. In certain embodiments, the tanning agent is an aluminum compound, alum, zirconium, titanium, iron salts, or a combination thereof. In particular embodiments, the tanning agent or agents are environmentally-friendly. By way example, in some embodiments, the tanning agent(s) do not include metals that are detrimental to human health and the environment such as chromium, lead, or arsenic. By way of further example, in some embodiments, the tanning agent(s) do not include organic chemicals that are detrimental to human health and the environment such as anthracene or formaldehyde.

In certain embodiments, the cultured leather comprises exogenous collagen. In certain embodiments, exogenous collagen is added at a final concentration of 1 μg/ml. In certain embodiments, exogenous collagen is added at a final concentration of 5 μg/ml. In certain embodiments, exogenous collagen is added at a final concentration of 10 μg/ml. In certain embodiments, exogenous collagen is added at a final concentration of 20 μg/ml. In certain embodiments, exogenous collagen is added at a final concentration of 30 μg/ml. In certain embodiments, exogenous collagen is added at a final concentration of 40 μg/ml. In certain embodiments, exogenous collagen is added at a final concentration of 50 μg/ml. In certain embodiments, the exogenous collagen is bovine. In certain embodiments, the exogenous collagen is porcine. In certain embodiments, the exogenous collagen is avian. In certain embodiments, the exogenous collagen is Type I. In certain embodiments, the exogenous collagen is Type I. In certain embodiments, the exogenous collagen is Type III. In certain embodiments, the exogenous collagen is Type IV. In certain embodiments, the exogenous collagen is Type V. In certain embodiments, the exogenous collagen is Type VI. In certain embodiments, the exogenous collagen is Type VII. In certain embodiments, the exogenous collagen is Type VIII. In certain embodiments, the exogenous collagen is Type IX. In certain embodiments, the exogenous collagen is Type X. In certain embodiments, the exogenous collagen is Type XI. In certain embodiments, the exogenous collagen is Type XII. In certain embodiments, the exogenous collagen is Type XIII In certain embodiments, the exogenous collagen is Type XIV. In certain embodiments, the exogenous collagen is Type XV. In certain embodiments, the exogenous collagen is Type XVI. In certain embodiments, the exogenous collagen is Type XVII. In certain embodiments, the exogenous collagen is Type XVIII. In certain embodiments, the exogenous collagen is a combination of one or more type of collagen.

In certain embodiments, the cultured leather comprises exogenous extracellular matrix (ECM) proteins other than collagen. In certain embodiments, the exogenous ECM protein is fibronectin. In certain embodiments, the exogenous ECM protein is laminin. In certain embodiments, the exogenous ECM protein is elastin. In certain embodiments, the exogenous ECM protein is a combination of one or more extracellular matrix proteins.

### Endogenous Source of Collagen

In certain embodiments, collagen is produced endogenously by the cells of the cultured leather. In certain embodiments, endogenous collagen synthesis is stimulated via mechanical conditioning. In certain embodiments, endogenous collagen synthesis is stimulated via the activation of signaling pathways. In certain embodiments, endogenous collagen synthesis is increased via increased gene expression using recombinant DNA technology.

Mechanical conditioning of cells increases collagen expression in cells as well as collagen remodeling and maturation. In some embodiments, mechanical stretching promotes collagen synthesis. In certain embodiments, cyclic strain promotes collagen synthesis. In certain embodiments, compression promotes collagen synthesis. In certain embodiments, the mechanical forces are external and generated from a non-cellular source. In other embodiments, the mechanical forces are internal and generated from cell-cell interactions or cell-environment interactions. Additionally, electrical stimulation of cells increases collagen expression. In certain embodiments, the electrical stimulation is pulsed. In certain embodiments, the electrical stimulation is directed only to the cells and not the extracellular matrix.

Recombinant DNA technology is used to increase cellular gene expression of collagen or a collagen variant. In certain embodiments, the cells of the cultured leather are induced to express collagen or a collagen variant through the delivery of genetic material encoding collagen. In certain embodiments, the genetic material is transfected into the cell via electroporation or chemical transfection. In certain embodiments, the genetic material is DNA. In certain embodiments, the genetic material is RNA. In certain embodiments, the cells are induced to produce collagen via the addition of activators of the signaling pathway for the production of one or more extracellular matrix proteins.

### Exogenous Source of Collagen

While endogenous collagen expression can be increased, in other embodiments, exogenous collagen is added to the cells of the cultured leather. In certain embodiments, collagen or a collagen variant is obtained commercially. In certain embodiments, collagen or a collagen variant is obtained by classical protein synthetic methods, including recombinant DNA technology or chemical synthesis. With recombinant DNA technology, the collagen or collagen variants are produced using cell-based or cell-free systems.

In certain embodiments of cell-based systems for protein synthesis, the cells that produce the protein are selected according to various factors, e.g., post-translational modifications, molecular folding, and multi-domain eukaryotic protein synthesis. In certain embodiments, DNA encoding the collagen or collagen variant is the template for protein production and is introduced into the host cell using various methods, such as electroporation and viral delivery. The DNA stably integrates into the host cell genome. In some cases, stable integration is not required. Many different host cells are available including those derived from bacteria, yeast, insect, and mammalian cells. Cell-based systems utilize bacterial hosts, eukaryotic hosts such as yeast cells, insect cells, and mammalian cells.

In certain embodiments of cell-free systems for protein synthesis, polypeptides and proteins are produced using biological machinery, e.g., ribosomes, aminoacyl-tRNA synthetases, translation initiation and elongation factors, nucleases, etc., without the use of living cells. In some embodiments, the biological machinery for cell-free protein expression is harvested from bacteria, insect cells, yeast, or mammalian cells. Reaction solutions comprising the biological machinery (e.g., ribosomes, aminoacyl-tRNA synthetases, translation initiation and elongation factors, nucleases, etc.), DNA template, amino acids, and other necessary supplements are incubated together to facilitate the in vitro translation of protein. Cell-free protein synthesis enables direct access to and control of the translation environment, which in certain cases, is advantageous for the optimization of protein production and incorporation of non-natural amino acids, amino acid analogues, modified amino acids, etc.

### Nucleotide Adhesion Molecules

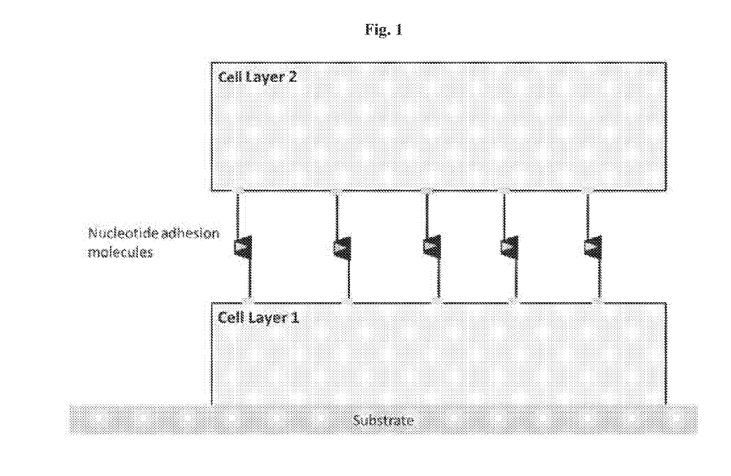

In certain embodiments, the cultured leather further comprises a nucleotide adhesion molecule. Adjacent layers of the contemplated cultured leather are held together in culture using nucleotides anchored to the cell membrane of each successive layer as shown in FIG. 1. Different adjacent layers have different complementary nucleotide adhesion molecules to provide for fast and sturdy assembly of cell layers in culture. Cellular assembly is dependent on cell-to-cell contact through connections such as gap junctions, tight junctions, and desmosomes. In certain embodiments, increased proximity of cells improves cell-to-cell contact by increasing the prevalence of cell-to-cell connections.

In certain embodiments, each adjacent layer comprises a nucleotide adhesion molecule. In certain embodiments, the nucleotide adhesion molecule comprises a membrane anchoring region, and an extracellular region. In certain embodiments, the extracellular region comprises a linker region and membrane distal adhesion region. Nucleotide adhesion molecules are described and exemplified in U.S. Patent Publication 2014/0294782 A1, which is incorporated by reference in its entirety.

In certain embodiments, the membrane anchoring region comprises one or more hydrophobic molecules that are capable of embedding in a cell membrane. In some embodiments, the membrane anchoring region is hydrophobic. In some embodiments, the membrane anchoring region is lipophilic. In some embodiments, the entire membrane anchoring region is hydrophobic. In some embodiments, the entire membrane anchoring region is lipophilic. In some embodiments, only a portion is lipophilic or hydrophobic. In some embodiments, the membrane anchoring region is amphiphilic. In many embodiments, the membrane anchoring region is such that it is energetically more favorable for the chain to be inserted into a membrane than be contained in solution. In certain embodiments, the membrane anchoring region comprises an alkyl chain. In some embodiments, the membrane anchoring region comprises an alkyl chain and an alkenyl, alkyl, aryl, or aralkyl chain. In certain embodiments, the alkenyl, alkyl, aryl, or aralkyl chain comprises 12-22 carbon atoms. In some embodiments, the alkyl chain comprises about 12-22 carbon atoms, and the alkenyl, alkyl, aryl, or aralkyl chain comprises about 12-22 carbon atoms. In some embodiments, the chains share the same number of carbon atoms. In other embodiments, one chain has between about 1 and about 10 fewer carbon atoms than the other chain. In some embodiments, one chain has about 1 fewer carbon atom than the other chain, about 2 fewer carbon atoms, about 3 fewer carbon atoms, about 4 fewer carbon atoms, about 5 fewer carbon atoms, about 6 fewer carbon atoms, about 7 fewer carbon atoms, about 8 fewer carbon atoms, about 9 fewer carbon atoms, or about 10 fewer carbon atoms. The membrane anchoring region, in various embodiments, comprises more than one alkenyl, aryl, or aralkyl chain, with each chain comprising 12-22 carbon atoms. In certain embodiments, the membrane anchoring region comprises an alkyl chain of at least 12 carbons. In certain embodiments, the membrane anchoring region comprises an alkyl chain of at least 14 carbons. In certain embodiments, the membrane anchoring region comprises an alkyl chain of at least 16 carbons. In certain embodiments, the membrane anchoring region comprises an alkyl chain of at least 18 carbons. In certain embodiments, the alkyl chain is saturated. In certain embodiments, the alkyl chain is unsaturated.

In certain embodiments, the extracellular region comprises a polynucleotide. In certain embodiments, the extracellular region comprises a single stranded DNA polynucleotide. In certain embodiments, the DNA polynucleotide comprises a linker region and a membrane distal adhesion region.

In certain embodiments, the extracellular region comprises a linker region. In certain embodiments, the linker region comprises a contiguous stretch of about 20 to about 3000 nucleotides. In many embodiments, the linker region is separated from the membrane distal end of the polynucleotide by about 10 nucleotides or more. In many embodiments, the linker region is separated from the membrane distal end of the polynucleotide by about 10 to about 2000 nucleotides or more. In some embodiments, the linker region is separated from the membrane distal end by about 5 to about 10 nucleotides, about 10 to about 20 nucleotides, about 20 to about 40 nucleotides, about 40 to about 60 nucleotides, about 60 to about 80 nucleotides, about 80 to about 100 nucleotides, about 100 to about 120 nucleotides, about 120 to about 140 nucleotides, about 140 to about 160 nucleotides, about 160 to about 180 nucleotides, about 180 to about 200 nucleotides, about 200 to about 225 nucleotides, about 225 to about 250 nucleotides, about 250 to about 275 nucleotides, about 275 to about 300 nucleotides, about 300 to about 350 nucleotides, about 350 to about 400 nucleotides, about 450 to about 500 nucleotides, about 500 to about 600 nucleotides, about 600 to about 700 nucleotides, about 700 to about 800 nucleotides, about 800 to about 900 nucleotides, about 1000 to about 1200 nucleotides, about 1200 to about 1400 nucleotides, about 1400 to about 1600 nucleotides, about 1600 to about 1800 nucleotides, about 1800 to about 2000 nucleotides. In many embodiments, the linker region comprises a contiguous stretch of about 5 to about 3000 identical nucleotides. In some embodiments, the linker region comprises a contiguous stretch of about 10 to about 2000 nucleotides comprising only two types of bases. In some embodiments, the linker region does not hybridize with any contiguous stretch of at least about 10 nucleotides that are distal to the linker region in the polynucleotide. In some embodiments, the linker region does not hybridize with any contiguous stretch of about 10 to about 500 nucleotides that are distal to the linker region in the polynucleotide.

In certain embodiments, the linker region comprises a contiguous stretch of about 5 to about 3000 identical nucleotides. In some embodiments, the contiguous stretch comprises about 5 to about 10 nucleotides, about 10 to about 20 nucleotides, about 20 to about 40 nucleotides, about 40 to about 60 nucleotides, about 60 to about 80 nucleotides, about 80 to about 100 nucleotides, about 100 to about 120 nucleotides, about 120 to about 140 nucleotides, about 140 to about 160 nucleotides, about 160 to about 180 nucleotides, about 180 to about 200 nucleotides, about 200 to about 225 nucleotides, about 225 to about 250 nucleotides, about 250 to about 275 nucleotides, about 275 to about 300 nucleotides, about 300 to about 350 nucleotides, about 350 to about 400 nucleotides, about 450 to about 500 nucleotides, about 500 to about 600 nucleotides, about 600 to about 700 nucleotides, about 700 to about 800 nucleotides, about 800 to about 900 nucleotides, about 1000 to about 1200 nucleotides, about 1200 to about 1400 nucleotides, about 1400 to about 1600 nucleotides, about 1600 to about 1800 nucleotides, about 1800 to about 2000 nucleotides, about 2000 to about 2250 nucleotides, about 2250 to about 2500 nucleotides, about 2500 to about 2750 nucleotides, about 2750 to about 3000 nucleotides or more. In particular embodiments, the contiguous stretch comprises thymine nucleotides. In other embodiments, the contiguous stretch comprises adenine nucleotides. In still other embodiments, the contiguous stretch comprises cytosine nucleotides. In still other embodiments, the contiguous stretch comprises guanine nucleotides. In yet other embodiments, the contiguous stretch comprises uracil nucleotides.

In certain embodiments, the extracellular region comprises a membrane distal adhesion region. In certain embodiments, the extracellular region comprises the polynucleotide region distal to the linker region and before the membrane distal end comprises a membrane distal adhesion region. In certain embodiments, the membrane distal adhesion region hybridizes to a polynucleotide, fluorophore, or pharmaceutical composition. In certain embodiments, the membrane distal adhesion region hybridizes to a polynucleotide present in another membrane anchored polynucleotide. In certain embodiments, the hybridization is between the membrane distal adhesion regions of the two membrane anchored polynucleotides. In certain embodiments, the hybridization is strict hybridization. In certain embodiments, the sequence of the membrane distal adhesion region does not hybridize with any other region of the polynucleotide. In certain embodiments, the membrane distal adhesion region comprises about 5 to about 3000 nucleotides. In particular embodiments, it comprises about 5 to about 10 nucleotides, about 10 to about 20 nucleotides, about 20 to about 40 nucleotides, about 40 to about 60 nucleotides, about 60 to about 80 nucleotides, about 80 to about 100 nucleotides, about 100 to about 120 nucleotides, about 120 to about 140 nucleotides, about 140 to about 160 nucleotides, about 160 to about 180 nucleotides, about 180 to about 200 nucleotides, about 200 to about 225 nucleotides, about 225 to about 250 nucleotides, about 250 to about 275 nucleotides, about 275 to about 300 nucleotides, about 300 to about 350 nucleotides, about 350 to about 400 nucleotides, about 450 to about 500 nucleotides, about 500 to about 600 nucleotides, about 600 to about 700 nucleotides, about 700 to about 800 nucleotides, about 800 to about 900 nucleotides, about 1000 to about 1200 nucleotides, about 1200 to about 1400 nucleotides, about 1400 to about 1600 nucleotides, about 1600 to about 1800 nucleotides, about 1800 to about 2000 nucleotides, about 2000 to about 2250 nucleotides, about 2250 to about 2500 nucleotides, about 2500 to about 2750 nucleotides, about 2750 to about 3000 nucleotides or more. In certain embodiments, the membrane distal adhesion region comprises (CAGT), and/or (ACTG)n, where n is an integer equal to or greater than 1. In some embodiments, n is 1. In other embodiments, n is between 1 and 20. In certain embodiments, n is between 1 and 10, or more preferably between 1 and 5.

In certain embodiments, the cultured leather further comprises a first and a second nucleotide adhesion molecule. In certain embodiments, the second nucleotide adhesion molecule is the same as the first except that the membrane distal adhesion region is different and comprises a polynucleotide sequence complimentary to the membrane distal adhesion region of the first nucleotide adhesion molecule.

In certain embodiments, the nucleotide adhesion molecule comprises a DNA signature that is amplifiable by the polymerase chain reaction. In certain embodiments, the DNA signature contains a non-naturally occurring continuous sequence of 10 or more nucleotides. In certain embodiments, the DNA signature contains a non-naturally occurring continuous sequence of 20 or more nucleotides. In certain embodiments, the DNA signature contains a non-naturally occurring continuous sequence of 30 or more nucleotides. In certain embodiments, the polynucleotide comprising the DNA signature contains primer binding sites. In a particular embodiment, the DNA signature is used to verify that a consumer product was made from cultured leather fabricated by the methodologies described herein.

By way of example, described herein are methods of identifying the methodology used to fabricate a cultured leather product, the method comprising: extracting nucleic acid from a sample of the cultured leather of the product; subjecting the nucleic acid to an amplification procedure using a primer specific to a known DNA signature, the DNA signature comprising a particular non-naturally occurring nucleic acid sequence, the particular non-naturally occurring nucleic acid sequence introduced into the cultured leather in the fabrication process; testing for an amplified presence of the DNA signature; and identifying the methodology used to fabricate the cultured leather product based on presence or absence of the amplified DNA signature. In some embodiments, the sample is obtained from a portion of the product provided specifically to allow verification of the methodology of fabrication. In some embodiments, wherein the amplification procedure is polymerase chain reaction (PCR). In some embodiments, the particular non-naturally occurring nucleic acid sequence is at least 10, 20, or 30 nucleic acids in length. In further embodiments, the particular non-naturally occurring nucleic acid sequence contains primer binding sites.

By way of further example, described herein are methods of identifying a leather product as a cultured leather product, the method comprising: extracting nucleic acid from a sample of the leather of the product; subjecting the nucleic acid to an amplification procedure using a primer specific to a known DNA signature, the DNA signature comprising a particular non-naturally occurring nucleic acid sequence, the particular non-naturally occurring nucleic acid sequence introduced into cultured leather in a fabrication process; testing for an amplified presence of the DNA signature; and identifying the leather product as a cultured leather product based on presence or absence of the amplified DNA signature. In some embodiments, the sample is obtained from a portion of the product provided specifically to allow verification of the leather product as a cultured leather product. In some embodiments, the amplification procedure is polymerase chain reaction (PCR). In some embodiments, the particular non-naturally occurring nucleic acid sequence is at least 10, 20, or 30 nucleic acids in length. In further embodiments, the particular non-naturally occurring nucleic acid sequence contains primer binding sites.

### Structural Support

Extracellular matrix proteins serve to provide structural integrity in tissues. The cellular solution comprising cells and any additional proteins is seeded into the scaffold. When engineering tissues, in some embodiments, scaffolds are used to provide structural support or to assist in cellular alignment.

In some embodiments, the scaffold is fabricated from biodegradable polymers (e.g., poly-lactic acid, polyglycolic acid, poly-di-lactic-co-glycolic acid, etc.). In some embodiments, the scaffold is functionalized with additionally moieties to render it bioactive (e.g., peptides, lipids, nucleic acids, or a pharmaceutical compound). In some embodiments, the scaffold is derived from de-cellularized tissue. In some embodiments, the de-cellularized tissue is mammalian. In some embodiments, the de-cellularized tissue is from a plant. The choice of material influences the stiffness of the scaffold and the engineered tissue. Additionally, the structure of the scaffold contributes to the stiffness of the scaffold. For example, changing the size of the pores in the scaffold alters the stiffness of the scaffold.

### Physical Characteristics of the Cultured Leather

The cultured leather of the present disclosure is intended to mimic the appearance and feel of natural leather. In certain embodiments, the cultured leather has a moisture content similar to that of natural leather. In certain embodiments, the moisture content of the cultured leather is less than 40%. In certain embodiments, the moisture content of the cultured leather is less than 30%. In certain embodiments, the moisture content of the cultured leather is less than 20%. In certain embodiments, the moisture content of the cultured leather is greater than 5%. In certain embodiments, the moisture content of the cultured leather is greater than 10%. In certain embodiments, the moisture content of the cultured leather is greater than 15%. In certain embodiments, the moisture content of the cultured leather is greater than 20%. In certain embodiments, the moisture content of the cultured leather is between 5% and 40%. In certain embodiments, the moisture content of the cultured leather is between 10% and 35%. In certain embodiments, the moisture content of the cultured leather is between 10% and 30%.

The cultured leather of the present disclosure is intended to mimic the appearance and feel of leather sourced from dead animal skins. In certain embodiments, the cultured leather has a tensile strength of leather prepared from dead animal skins. In certain embodiments, the cultured leather has a tensile strength of at least 50 kgf/cm². In certain embodiments, the cultured leather has a tensile strength of at least 75 kgf/cm². In certain embodiments, the cultured leather has a tensile strength of at least 100 kgf/cm². In certain embodiments, the cultured leather has a tensile strength of at least 125 kgf/cm². In certain embodiments, the cultured leather has a tensile strength of at least 150 kgf/cm². In certain embodiments, the cultured leather has a tensile strength of at least 175 kgf/cm². In certain embodiments, the cultured leather has a tensile strength of at least 200 kgf/cm². In certain embodiments, tensile strength testing is carried out according to American Society for Testing and Materials (ASTM) standards.

Synthetic Leather vs. Natural Leather

Synthetic leather is distinguishable from natural leather in some aspects. First, the cellular composition of natural leather is different from the cellular composition of natural leather. The genetic profile of cells derived from pluripotent stem cells is different from the genetic profile of their analogous adult cells. The difference in genetic profile yields differences in gene expression profiles, which ultimately leads to differences in protein synthesis, protein structure, and protein function.

Additionally, the ratio of cell types in natural skin is different from the ratio of cell types in engineered skin tissue. As a result, the differentiated cells do not spontaneously form the sub-structures found in natural skin such as the sebaceous glands, vasculature, or hair follicles. The formation of engineered tissue relies on many factors including the alignment of cells, the interaction of cells, the expression and secretion of extracellular matrix by the cells, and the maturation of the extracellular matrix proteins. These factors affect the properties of the engineered tissue as well as the cultured leather.

### Methods of Making Cultured Leather

Disclosed herein are methods of making cultured leather. Skin cells such as fibroblasts, keratinocytes, melanocytes, or a combination thereof that have been sampled from a donor animal are used for the production of the cultured leather. In certain embodiments, the sampling does not kill the animal. In certain embodiments, the sampling does not cause pain to the animal. In certain embodiments, the cells are pluripotent stem cells differentiated into fibroblasts, keratinocytes, and/or melanocytes. In certain embodiments, the cells are induced pluripotent stem cells differentiated into fibroblasts, keratinocytes, or melanocytes. In certain embodiments, induced pluripotent stem cells are created by chemical, gene, or viral treatments.

Cultured leather is fabricated by an additive manufacturing process. A piece of finished cultured leather will comprise collagen released by several layers of cells. Layers are laid down successively. The first layer is laid onto a substrate that comprises a tissue culture compatible glass, plastic, or mesh. In certain embodiments, the substrate is coated to facilitate adhesion of the first layer with a substance such as collagen or lysine. In certain embodiments, the substrate is biodegradable and/or dissolvable after addition of the first layer. The first layer is functionalized with a nucleotide adhesion molecule either before or after addition to the substrate. The successive layers are laid down each layer functionalized with a nucleotide adhesion molecule that promotes adhesion with the layer laid down before it. In certain embodiments, adhesion is promoted by complimentary base pairing of the nucleotide adhesion molecules. In certain embodiments, cells are allowed to adhere completely before addition of a successive layer. In certain embodiments, cells are allowed to adhere for at least 10, 20, 30, 40, 50, 60, 90, 120, 180, or 240 minutes, including increments therein, before addition of a successive layer.

Many methods of depositing the cells of each layer are suitable. In certain embodiments, cells of each layer are deposited by application of a liquid, cell-containing suspension. In certain embodiments, cells of each layer are deposited by bioprinting. In further embodiments, the bioprinting technology comprises ink-jetting or spraying of a liquid or semi-liquid cell-containing solution. In still further embodiments, the bioprinting technology comprises application or extrusion of a semi-solid or solid cell-containing composition. In certain embodiments, cells of each layer are deposited by patterning cells on a surface of a substrate. In further embodiments, the patterning comprises disposing a pattern of nucleic acids on a surface of a substrate, and contacting the patterned nucleic acids under hybridization conditions with a first suspension of cells, where cells of the first suspension include cell surface-attached nucleic acids complementary to the patterned nucleic acids, and where the cell surface-attached nucleic acids hybridize to the patterned nucleic acids to pattern the cells on the surface of the substrate. Methods of patterning cells on a surface of a substrate are described and exemplified in U.S. Patent Publication 2016/0010054 A1, which is incorporated by reference in its entirety.

After cell layers are attached to the substrate they are cultured using standard tissue culture techniques and media. Cells are cultured at about 5%, and at temperatures between 24 and 40 degrees Celsius. Preferably between 30 and 38 degrees Celsius. Cells are cultured in a rich liquid medium such as RPMI, DMEM, or specialized cell media. In certain embodiments, the cells are cultured in a serum-free media capable of supporting cell growth and attachment. In certain embodiments, cells are cultured for at least 24, 48, 72, 96, or 120 hours, including increments therein, before removal for processing. In certain embodiments, cells are cultured for at least 1, 2, 3, 4, 5, 6, 7, 8, 9, 10, 11, 12, 13, or 14 days before removal from the substrate for further processing.

Cells are removed from the substrate in any way that allows for the maintenance of the integrity of the layers. In certain embodiments, the cells are removed by enzymatic digestion. In certain embodiments, the cells are removed by physical means such as scraping or shaking. After removal of the cell layers the cells are processed by an additional processing step to yield a leather-like fabric. In general, leather processing steps are well known in the art. In certain embodiments, the additional processing step comprises drying, preservation, soaking, liming, fleshing, splitting, reliming, deliming, bating, degreasing, frizing, bleaching, pickling, depickling, tanning, dyeing, or any combination thereof. In certain embodiments, the leather is subjected to one or more crusting steps such as sammying, splitting, shaving, rechroming, neutralization, retanning, dyeing, fatliquoring, filling, stuffing, stripping, whitening, fixating, setting, drying, conditioning, milling, staking, buffing, or any combination thereof. In certain embodiments, the culture leather is subjected to one or more finishing steps comprising, oiling, brushing, padding, impregnation, buffing, spraying, roller coating, curtain coating, polishing, plating, embossing, ironing, glazing, tumbling or any combination thereof.

Articles Manufactured from Cultured Leather

The purpose intended for the cultured leather of the present disclosure is for the manufacture of consumer products. In certain embodiments, the consumer product is any product that is made using natural leather.

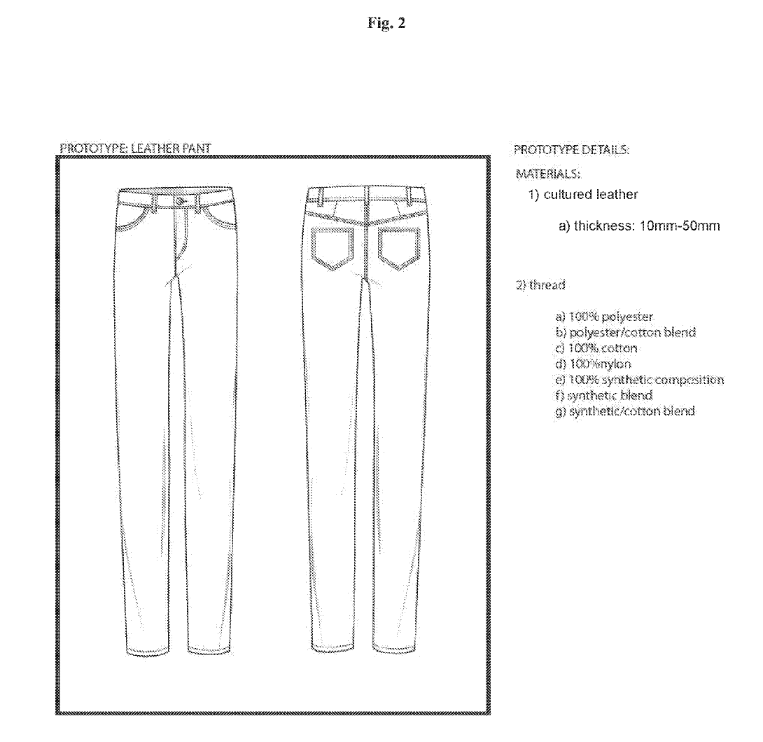

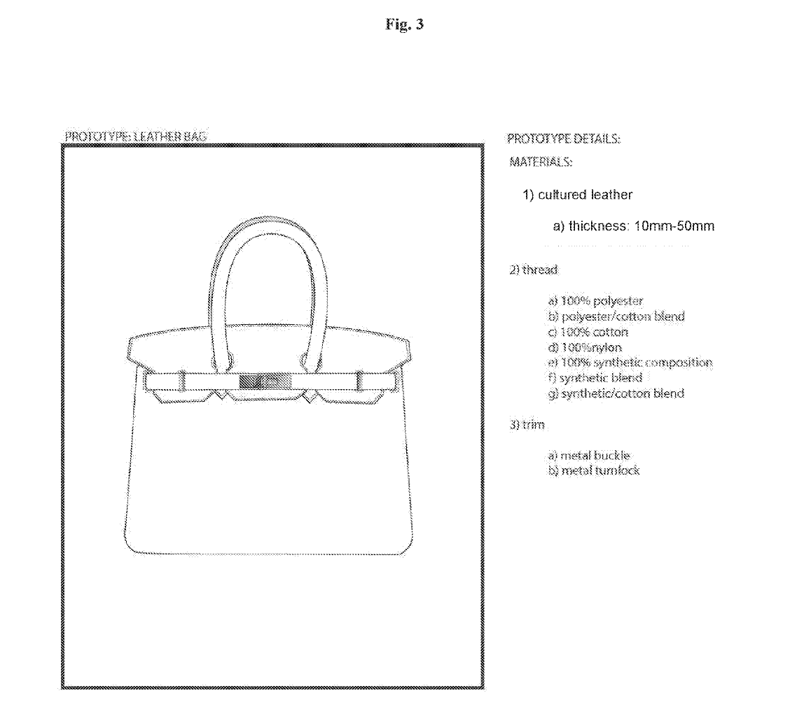

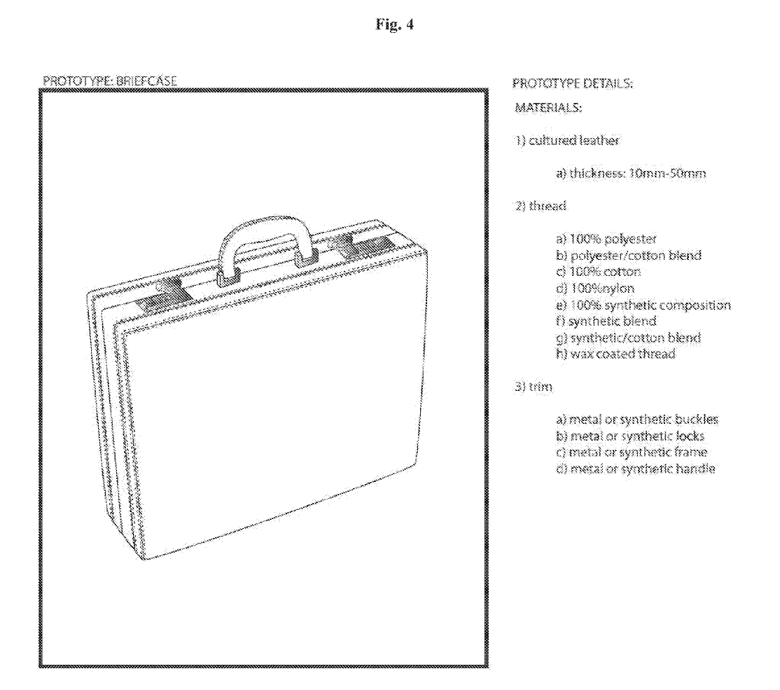

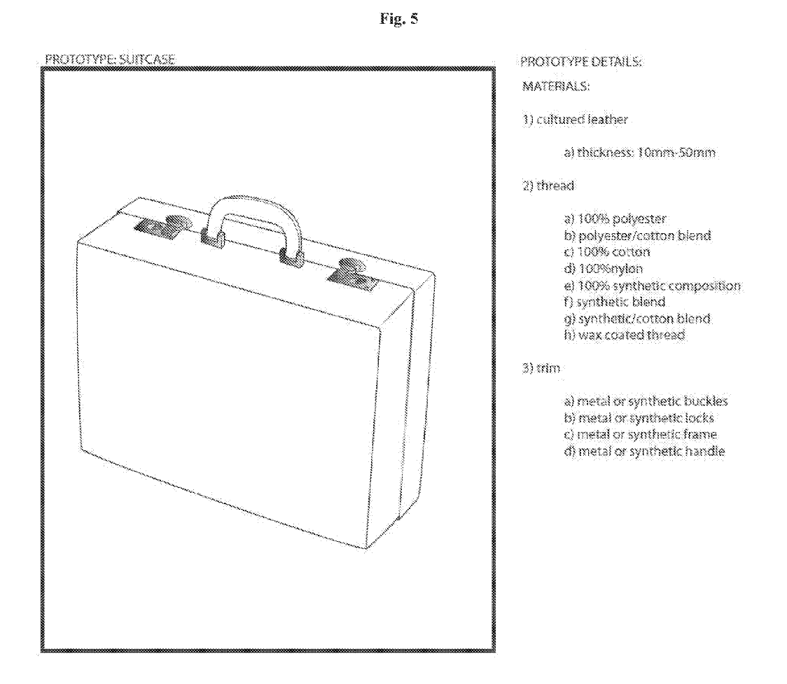

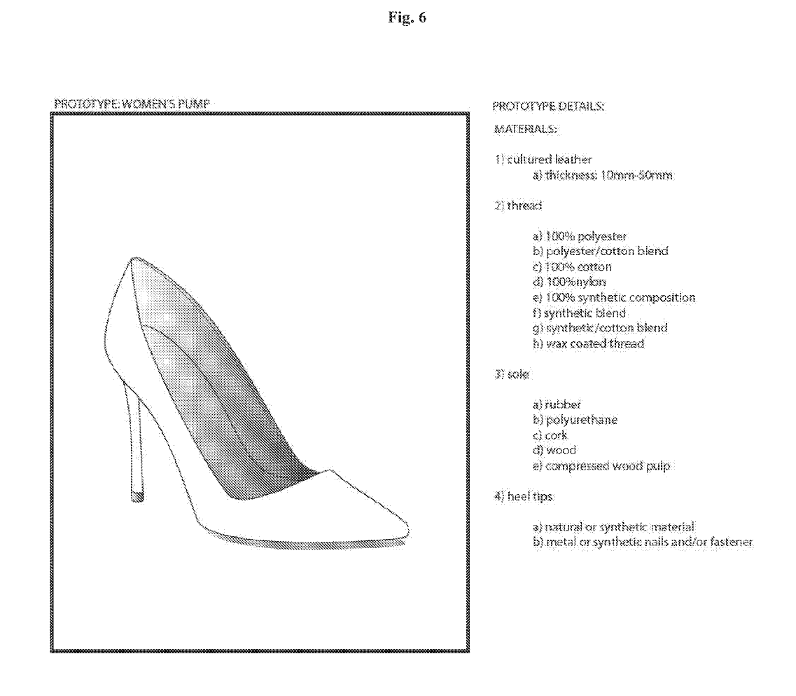

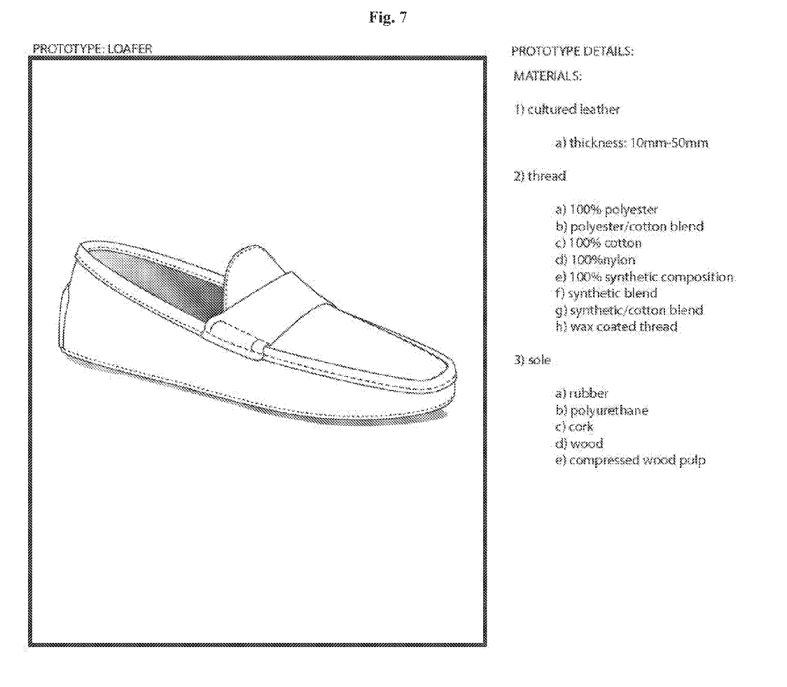

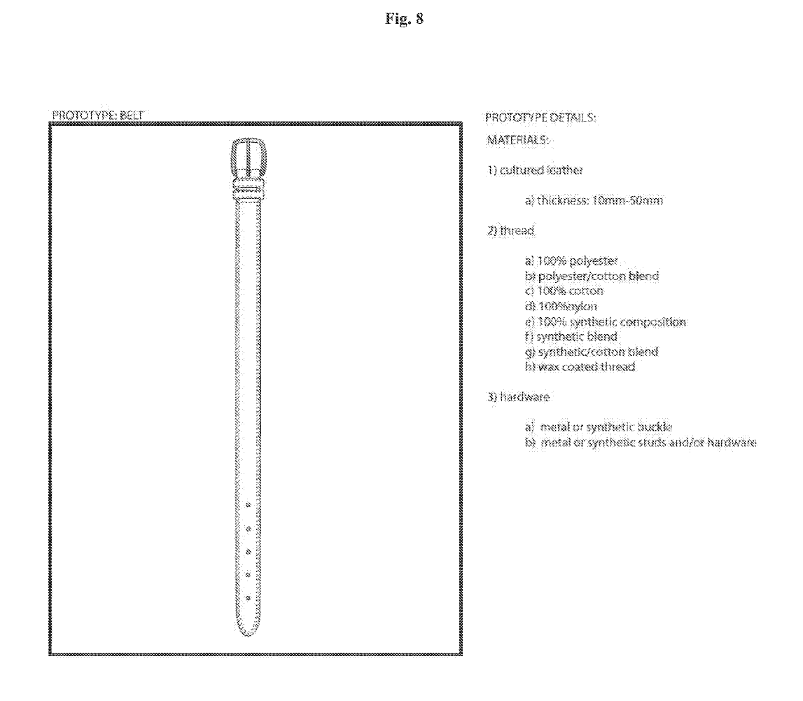

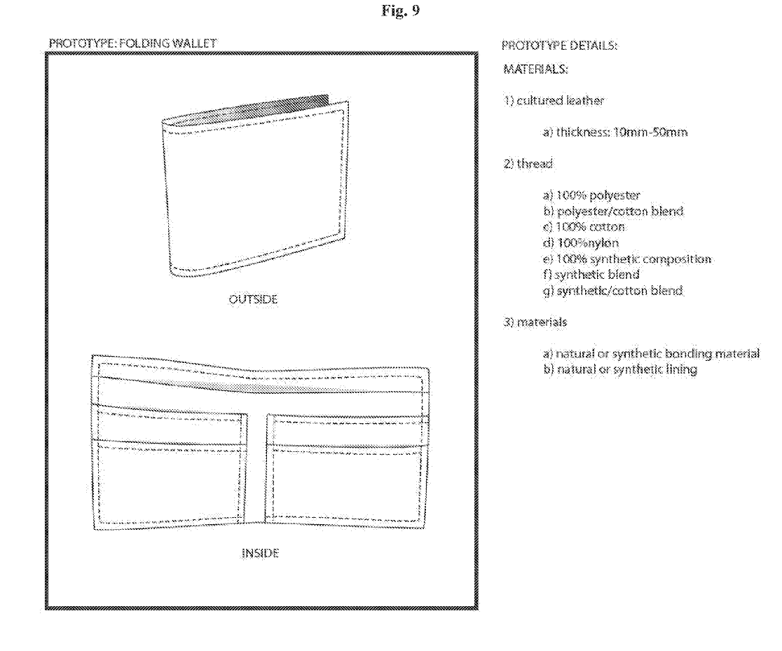

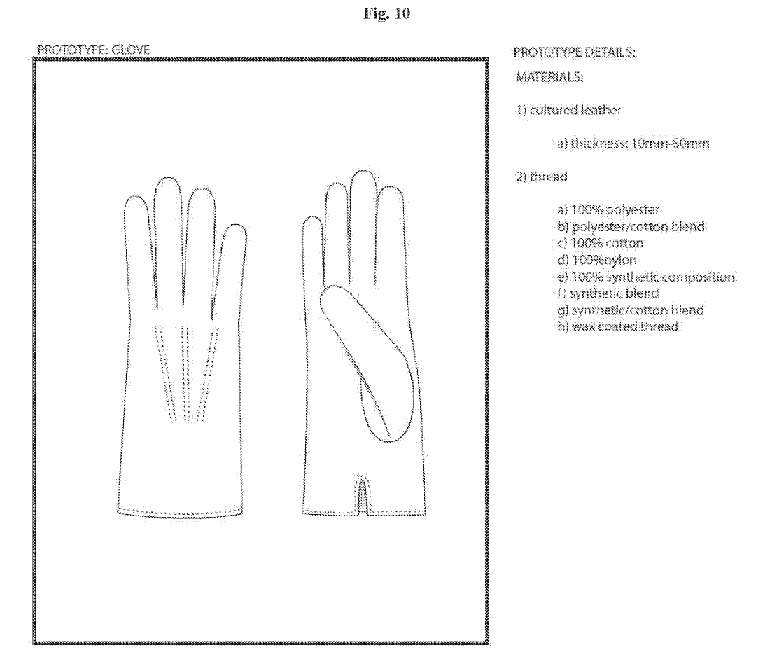

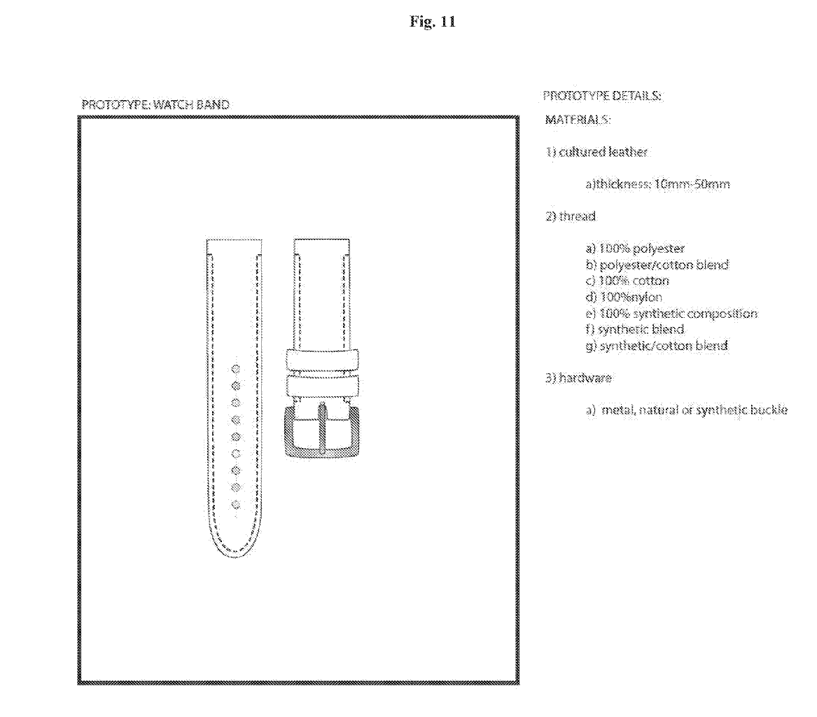

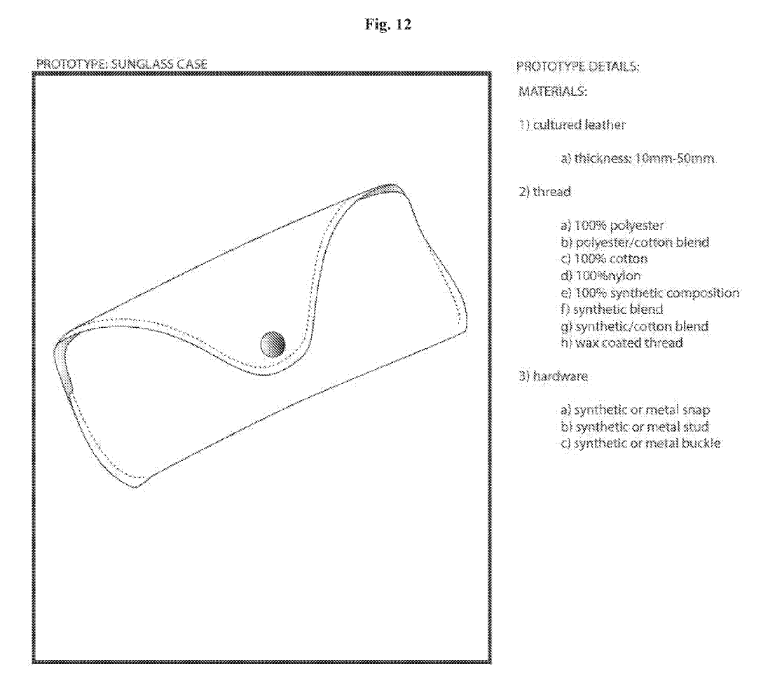

In some embodiments, the consumer products include fashion articles and/or articles of apparel. In various further embodiments, the consumer products include, by way of non-limiting examples, pants (see, e.g., FIG. 2), shorts, handbags (see, e.g., FIG. 3), satchels, briefcases (see, e.g., FIG. 4), backpacks, laptop computer bags, luggage (see, e.g., FIG. 5), luggage tags, totes, mobile phone cases, laptop computer cases, laptop computer coverings, tablet cases, shoes (e.g., heels (see, e.g., FIG. 6), sandals, slippers, loafers (see, e.g., FIG. 7), lace-ups, boots, booties, shoelaces, etc.), jackets (e.g., raincoats, motorcycle jackets, etc.), skirts, shirts (e.g., t-shirts, tanks, jerseys, etc.), undergarments, vests, chaps, belts (see, e.g., FIG. 8), wallets (see, e.g., FIG. 9), gloves (see, e.g., FIG. 10) including motorcycle gloves, hats, jewelry (e.g., bracelets, bangles, earrings, necklaces, watch bands (see, e.g., FIG. 11), etc.), body armor, helmets, decorations (e.g., tassels, etc.), sunglasses, and sunglass cases (see, e.g., FIG. 12).

In some embodiments, the consumer products include pet accessories. In various further embodiments, the consumer products include, by way of non-limiting examples, animal (dog) collars, animal (dog) leashes, animal (dog) apparel, and the like.

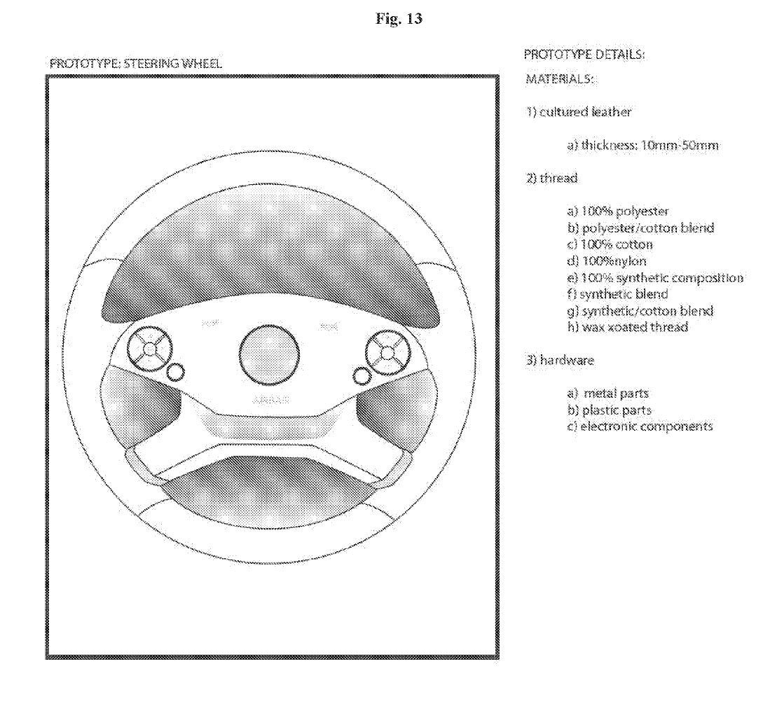

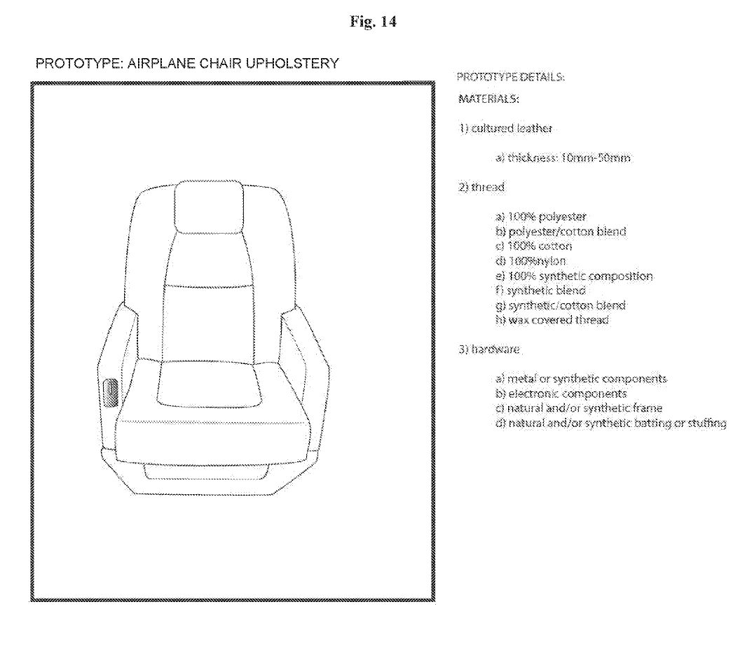

In some embodiments, the consumer products include vehicle components and appointments. In various further embodiments, the consumer products include, by way of non-limiting examples, steering wheel covers (see, e.g., FIG. 13), automotive dashboard, automotive arm rests, automotive captain's chairs, automotive seats and benches, automotive interior trim, airplane seats (see, e.g., FIG. 14), airplane interior trim, boat seating, motorcycle seats, and the like.

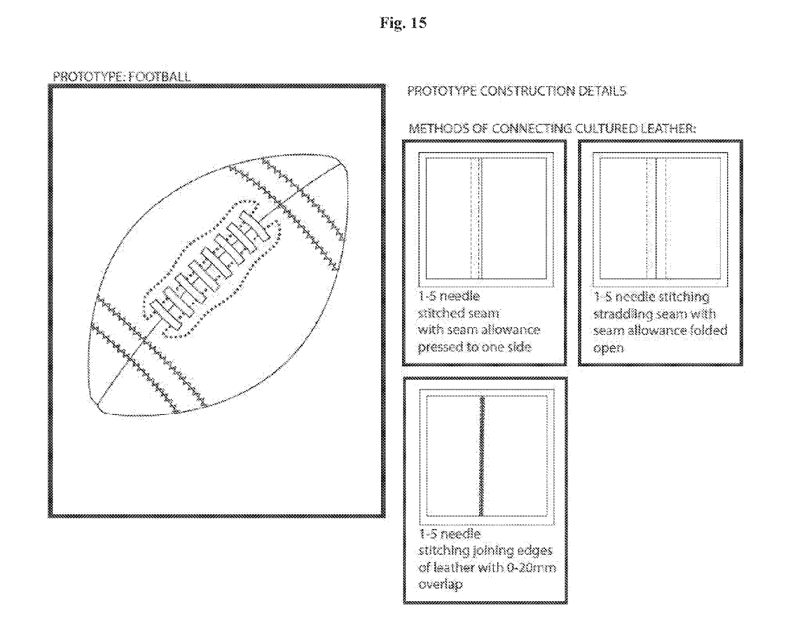

In some embodiments, the consumer products include sports and athletic equipment. In various further embodiments, the consumer products include, by way of non-limiting examples, footballs (see, e.g., FIG. 15), baseballs, softballs, soccer balls, volleyballs, cricket balls, rugby balls, handballs, cleats, climbing shoes, cycling shoes, bicycle seats, baseball gloves, golf gloves, boxing gloves, ski/snowboard gloves, yoga mats, Pilates equipment, punching bags, gym benches, equine sport equipment, horse saddlery and equipment, pommel horses, balance beams, tents, tepees, wigwams, yurts (housing), kayaks, falconry jesses, archer bracers, bullwhips, horse hoof boots, boxing speed bags, sword grips, knife grips, sword sheathes, arrow quivers, knife sheaths, gun holsters, gun sleeves, and the like.

In some embodiments, the consumer products include, by way of non-limiting examples, leather-accented tools, carpentry tool belts, carpentry nail bags, and the like.

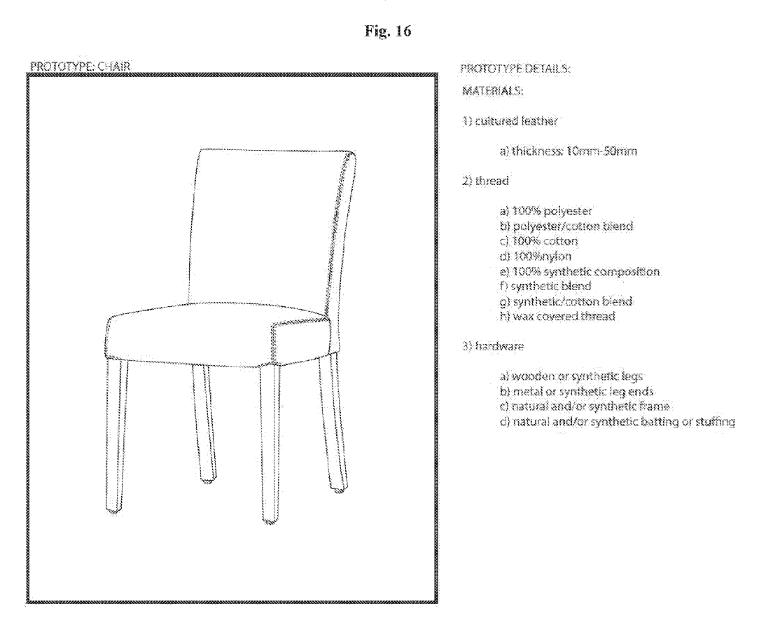

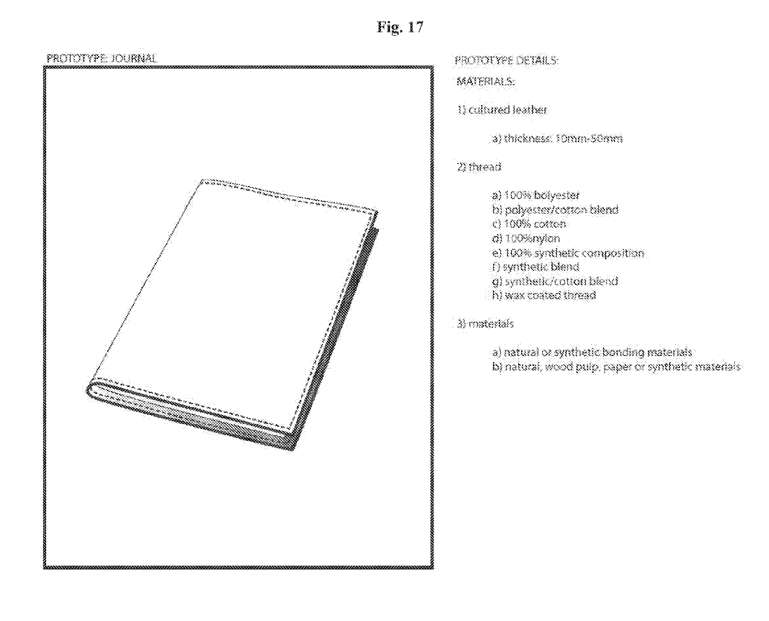

In some embodiments, the consumer products include furniture (e.g., upholstered beds, headboards, chairs (see, e.g., FIG. 16), cushions, pillows, draperies, etc.). In some embodiments, the consumer products include, flooring. In some embodiments, the consumer products include, housewares (e.g., drink coasters, valet trays, picture frames, flasks, gourds, upholstered stuffed animals, rugs, etc.). In some embodiments, the consumer products include, by way of non-limiting examples, books, book covers, book bindings, journals (see, e.g., FIG. 17), journal covers, and leather drawing pads.

In some embodiments, the consumer products include musical instruments (e.g., drums, banjos, guitar straps, etc.).

In some embodiments, the consumer products include parchment and/or vellum.

### EXAMPLES

The following illustrative example is representative of embodiments of the subject matter described herein and are not meant to be limiting in any way.

### Example 1—Method of Making Synthetic Leather from Bovine Derived Pluripotent Stem Cells

The examples described herein are illustrative and not intended to limit the claims in any way. This example describes a method of making cultured leather. Tissue is engineered from differentiated bovine induced pluripotent stem (iPS) cells, nucleotide adhesion molecules, and an exogenous source of collagen. The engineered tissue is further processed to develop cultured leather.

### Developing Bovine Induced Pluripotent Stem Cells

In order to develop bovine iPS cells, a blood sample is first obtained from a bovine animal. The cells are isolated from the plasma via Ficoll density centrifugation. To induce de-differentiation of the blood cells to iPS cells, the blood cells are induced to express Oct4, Sox2, cMyc, and Klf4 using methods commonly utilized by one skilled in the art. Cells, which are successfully induced, divide and form colonies; these cells are the iPS cells. The iPS cells are further prepared by selecting and expanding colonies as adherent cultures on tissue culture plates coated with basal membrane protein. The iPS cells are tested to confirm their ability to propagate and differentiate into all three germ layers. Bovine iPS cells are characterized for gene expression and protein expression of iPS cell biomarkers such as SSEA-1, SSEA-3, SSEA-4, Tra-1-60, Oct4, and Nanog.

Differentiating iPS Cells into Keratinocyte-Like Cells

Subsequently, the iPS cells are differentiated into keratinocyte-like cells. Tissue culture plates are coated with collagen type I at 3 mg/mL and basement membrane mixture such as Matrigel or Geltrex hESC-qualified Reduced Growth Factor Basement Membrane Matrix or a similarly equivalent basal membrane mixture. The mixture for coating is prepared in Dulbecco's Modified Eagle Medium/Nutrient Mixture F-12 (DMEM/F12) by first preparing a 1:100 dilution of Geltrex in DMEM/F12 and then adding collagen type I to reach a final collagen concentration of 3 mg/mL. The coated plates are incubated at 37° C. for 1 hr.

To prepare the first layer of cells, a confluent plate of iPS cells is dissociated using one or more dissociation agents including ethylenediaminetetraacetic acid (EDTA), Dispase, or Accutase. First, the media is aspirated from the dish of iPS cells and replaced with 1 mL of 100 nM EDTA. The cells are incubated with EDTA at 37° C. for 3-7 mn or until the cells are rounded but not yet detached. The EDTA is aspirated and the cells are resuspended in iPS cell media and replated at a ratio of 1:5 on a tissue culture dish, pre-coated with basement membrane mixture. The iPS cells are cultured in N2B27 medium, which consists of DMEM/F12, Neurobasal medium, 0.1 mM nonessential amino acids, 1 mM glutamine, 55 μM 2-mercaptoethanol, N2 supplement, B27 supplement, 50 μg/mL ascorbic acid, 0.05% bovine serum albumin, 50 U/mL penicillin-streptomycin, 100 ng/mL basic FGF, and 10 μM Y27632. The cells are incubated overnight in the 37° C. tissue culture incubator.

On the following day, differentiation protocol can commence if the iPS cells formed colonies. The differentiation process of iPS cells into keratinocyte-like cells is controlled by the media composition. The basal media for differentiation is defined by keratinocyte serum-free medium (DKSFM) sold by Gibco and supplemented by 50 U/mL penicillin-streptomycin. On day 1 of differentiation, the media is replaced with DKSFM containing 1 μM retinoic acid and 25 ng/mL BMP4. The cells are incubated for 48 hours. On day 3, the media is replaced with fresh DKSFM containing 1 μM retinoic acid and 25 ng/mL BMP4. On day 5, the media is replaced with fresh DKSFM without retinoic acid or BMP4. The cells are incubated in DKSFM for ten days, replacing media every other day. On day 14, the media is replaced with epidermal keratinocyte medium containing 50 U/mL penicillin-streptomycin. On day 24, cells that migrate away from the outgrown iPS cell colony exhibit keratinocyte-like phenotype and start expressing p63, a master regulator required for the commitment of the ectoderm to a keratinocyte fate, and Krt14.

Enrichment of keratinocyte-like cells is obtained through the rapid attachment on collagen-coated plates. Tissue culture plates are coated with collagen type IV at 7 μg/ml in 0.25% acetic acid and 30 μg/ml of collagen type I. The plates are incubated at room temperature for 1 hour. Then the collagen is aspirated and the plates rinsed with sterile 1× with PBS and 1× with distilled H₂O. The differentiated cells are passaged by incubating the cells with Accutase for 5 mn, dislodging the cells with additional keratinocyte medium, and resuspending the cells in keratinocyte medium. The cells are plated on the collagen-coated plates for 15-30 minutes. Any floating cells are aspirated and the remaining cells are expanded at 37° C., changing the medium every other day. When the plate reaches confluence, the cells are passaged at a 1:4 ratio onto new collagen-coated plates. After 2 or 3 passages, the culture consists of 90% Krt14 positive cells exhibiting a keratinocyte-like phenotype.

### Engineering Skin-Like Tissue

Engineered skin tissue is prepared with a mixture comprising of the differentiated keratinocyte-like cells, a collagen mixture, and nucleotide adhesion molecules. Keratinocyte-like cells are dissociated from the plates with Accutase (by incubating with Accutase for 5 mn, dislodging the cells with additional keratinocyte medium, and resuspending the cells in keratinocyte medium). The total number of cells is then counted. A first solution is prepared with cells (10 million cells per milliliter of keratinocyte media), collagen type I (10 μg/ml), and the first nucleotide adhesion molecule. The first nucleotide adhesion molecule is prepared in keratinocyte media. A second solution is prepared with cells (10 million cells per milliliter of keratinocyte media), collagen type I (10 μg/ml), and the second nucleotide adhesion molecule. The sequence of the second nucleotide adhesion molecule is complementary to the sequence of the first nucleotide adhesion molecule, thus allowing for complementary pairing between the first and second layers of cells.

The first solution is applied to a basement membrane coated substrate and incubated for 15 minutes to allow for the collagen to form a gel. Subsequently, the second solution is applied over the first layer. These solutions are applied successively and alternatively. The application is manual but in some embodiments the layers are applied using 3D printing technology. Once the layers reach a thickness of at least 5 mm, the tissue is detached and processed to form a leather product.

### Processing Cultured Leather

The engineered tissue is processed into cultured leather via methods that convert the protein of the cultured tissue into a stable material. This process is referred to as tanning and involves cross-linking the proteins and is achieved by the application of chromium. The cultured tissue is detached from the substrate using Dispase (10 mn incubation at 37° C.) and transferred to a solution of chromium tannin and incubated at room temperature for 15 minutes. Then the culture leather is submerged in magnesium oxide overnight at 45° C. at pH 3.5-4.0. Because the chrome tanning process is acidic, the cultured leather is neutralized with sodium bicarbonate and sodium formate or acetate. Then, the cultured leather is retanned with synthetic tanning agents such as syntans, resins, and natural tannins. The cultured leather is dyed to the required shade and fatliquored such that natural and synthetic oils are absorbed by the cultured leather. The cultured leather is then dried using a variety of methods including vacuum drying, hang drying, and stretch drying.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

1. A cultured leather comprising, a plurality of layers each layer comprising cells and extracellular collagen, wherein each layer of the plurality of layers is coupled to an adjacent layer by a nucleotide adhesion molecule, wherein the cultured leather comprises a moisture content less of less than 40%.

2. The cultured leather of claim 1, wherein the cells comprise fibroblasts.

3. The cultured leather of claim 1, wherein the cells comprise keratinocytes.

4. The cultured leather of claim 1, wherein the cells comprise induced pluripotent stem cells (iPSC).

5. The culture leather of claim 1, wherein the cells comprise pluripotent stem cell-derived skin-like cells.

6. The cultured leather of claim 1, wherein any of the cells originate from any one or more of a mammal, a bird, a fish, or a reptile.

7. The cultured leather of claim 1, wherein the extracellular collagen is cross-linked.

8. The cultured leather of claim 1, wherein the nucleotide adhesion molecule comprises:
a) a membrane anchoring region comprising a long alkyl chain of at least 12 carbon atoms; and
b) a polynucleotide having a membrane distal end and a membrane proximal end, wherein said polynucleotide is conjugated to said membrane anchoring region at the membrane proximal end wherein said polynucleotide is at least 50 nucleotides and comprises:
i) a linker region comprising a contiguous stretch of at least about 20 nucleotides; and
ii) a membrane distal adhesion region comprising at least 10 nucleotides and positioned distal to the linker region, wherein the linker region is not hybridizable to the membrane distal adhesion region.

9. The cultured leather of claim 8, wherein the nucleotide adhesion molecule comprises a DNA signature, wherein the DNA signature is able to be amplified after the tanning process.

10. The cultured leather of claim 1, wherein the moisture content is between 10% and 30%.

11. (canceled)

12. (canceled)

13. A consumer product comprising the cultured leather of claim 1.

14. The consumer product of claim 13, wherein the consumer product comprises a wallet, a purse, a watch band, a jacket, a shoe, a mobile phone case, a brief case, a piece of luggage, a glove, a belt, or a pair of pants.

15. A method of preparing a cultured leather comprising:
a) culturing a plurality of layers, each layer comprising cells, wherein each layer of the plurality of layers is coupled to an adjacent layer by a nucleotide adhesion molecule; and
b) contacting the plurality of layers with at least one tanning agent;
wherein the resulting cultured leather comprises a moisture content of less than 40%.

16. The method of claim 15, wherein the cells comprise fibroblasts.

17. The method of claim 15, wherein the cells comprise keratinocytes.

18. The method of claim 15, wherein the cells comprise induced pluripotent stem cells (iPSC).

19. The method of claim 15, wherein any of the cells originate from any one or more of a mammal, a bird, a fish, or a reptile.

20. The method of claim 15, wherein the collagen is cross-linked by the tanning agent.

21. (canceled)

22. (canceled)

23. The method of claim 15, wherein the nucleotide adhesion molecule comprises:
a) a membrane anchoring region comprising a long alkyl chain of at least 12 carbon atoms; and
b) a polynucleotide having a membrane distal end and a membrane proximal end, wherein said polynucleotide is conjugated to said membrane anchoring region at the membrane proximal end wherein said polynucleotide is at least 50 nucleotides and comprises:
i) a linker region comprising a contiguous stretch of at least about 20 nucleotides; and
ii) a membrane distal adhesion region comprising at least 10 nucleotides and positioned distal to the linker region, wherein the linker region is not hybridizable to the membrane distal adhesion region.

24. The method of claim 15, wherein the nucleotide adhesion molecule comprises a DNA signature, wherein the DNA signature is able to be amplified after the tanning process.

25. The method of claim 15, wherein the moisture content of the resulting cultured leather is between 10% and 30%.

26. (canceled)

27. (canceled)

28. The method of claim 15, further comprising using the cultured leather in the manufacture of a consumer product, and wherein the consumer product comprises a wallet, a purse, a watch band, a jacket, a shoe, a mobile phone case, a brief case, a piece of luggage, a glove, a belt, or a pair of pants.

29. A method of identifying the methodology used to fabricate a cultured leather product, the method comprising:
a. extracting nucleic acid from a sample of the cultured leather of the product;
b. subjecting the nucleic acid to an amplification procedure using a primer specific to a known DNA signature, the DNA signature comprising a particular non-naturally occurring nucleic acid sequence, the particular non-naturally occurring nucleic acid sequence introduced into the cultured leather in the fabrication process;
c. testing for an amplified presence of the DNA signature; and
d. identifying the methodology used to fabricate the cultured leather product based on presence or absence of the amplified DNA signature.

30. (canceled)

31. (canceled)

32. (canceled)

33. (canceled)

34. A method of identifying a leather product as a cultured leather product, the method comprising:
a. extracting nucleic acid from a sample of the leather of the product;
b. subjecting the nucleic acid to an amplification procedure using a primer specific to a known DNA signature, the DNA signature comprising a particular non-naturally occurring nucleic acid sequence, the particular non-naturally occurring nucleic acid sequence introduced into cultured leather in a fabrication process;
c. testing for an amplified presence of the DNA signature; and
d. identifying the leather product as a cultured leather product based on presence or absence of the amplified DNA signature.

35. (canceled)

36. (canceled)

37. (canceled)

38. (canceled)
